(19) United States
(12) Patent Application Publication
(10) Pub. No.: US 2019/0091287 A1
(43) Pub. Date: Mar. 28, 2019

(54) PLANT FRACTIONS HAVING ANTI-PATHOGENESIS PROPERTIES

(71) Applicants: Chitra Vasant Savangikar, Nashik, Maharashtra (IN); Vasant Anantrao Savangikar, Nashik, Maharashtra (IN)

(72) Inventors: Chitra Vasant Savangikar, Nashik, Maharashtra (IN); Vasant Anantrao Savangikar, Nashik, Maharashtra (IN)

(21) Appl. No.: 16/090,812

(22) PCT Filed: Apr. 1, 2017
(86) PCT No.: PCT/IN2017/050124

§ 371 Date: Oct. 2, 2018

(30) Foreign Application Priority Data
Apr. 2, 2016 (IN) 201621011729

(51) Int. Cl. A61K 38/16; A23J 1/00; A23J 3/14; A23L 33/105; A23L 33/185; A61K 9/20; A61P 3/10

(52) U.S. Cl. CPC A61K 38/168; A23J 1/006; A23J 3/14; A23V 2002/00; A23L 33/185; A61K 9/2095; A61P 3/10; A23L 33/105

(57) ABSTRACT

This invention comprises a method of using for anti-pathogenesis effect an improved composition and an improved composition comprising heat stable plant juice soluble plant proteins; the improvement comprising that the composition comprising heat stable plant juice soluble plant protein is essentially a water insoluble concentrated mass free from components more soluble in the plant juice, and enriched in heat stable proteins of the plant juice; dosage forms made from the composition for using for Adiponectin agonist effect for reducing the risk of one or more of a disorder selected from Type II diabetes, hyperlipidemia, atherosclerosis, hypertension, heart disease and obesity and for anti-microbial effect for reducing the risk of or treating the microbial infections in plants and animals, with or without synergistic interaction with other antimicrobial agents. This invention comprises a composition essentially characterized by Infra-red absorption spectrum provided therein.

### FIELD OF INVENTION

This invention pertains to Osmotin like proteins, compositions containing them and methods of making the compositions from plants. The invention also pertains to use of Osmotin like protein containing impure or semi-purified compositions of Osmotin or osmotin like proteins with other ingredients derived from the green leaves in the form of fractions made from plants for treatment of diseases and disorders that arise from deficiency of Adiponectin in a human being. Such diseases and disorders include type II diabetes, insulin resistance, hyperlipidemia, arteriosclerosis, and heart disease.

### BACKGROUND OF THE INVENTION

Singh et al (1987) (NARENDRA K. SINGH, CHARLES A. BRACKER, PAUL M. HASEGAWA, AVTAR K. HANDA, SCORR BUCKEL, MARK A. HERMODSON, ED PFANKOCH, FRED E. REGNIER, AND RAY A. BRESSAN* Plant Physiol. (1987) 85, 529-536) reported that specific alterations in gene expression of salt adapted cells of tobacco occur since several novel proteins are made by adapted cells including a predominant 26 kD protein. This protein was named by Singh et al as “Osmotin” because it is synthesized and accumulated by cells undergoing gradual osmotic adjustment to either salt or desiccation stress but is not induced by osmotic shock. Singh et al further add that Osmotin is a cationic protein with a pI2 value >8.2″. Singh et al also observe that in unstressed unadapted cells, an immunologically related 26 kD protein with a lower pI is also synthesized but is not accumulated under normal growth conditions. Osmotin accumulation is reported to depend on adjustment of the cells to NaCl or to water stress. The method for isolation described by Singh et al comprises homogenization of cold acetone washed cells of tobacco that were adapted to 25 g/L NaCl, (a) extracting them in a buffer containing no detergent and collecting the supernatant after centrifugation as Fraction I, and (b) extracting the cell pellet with extraction buffer containing a detergent and collecting the supernatant as Fraction II. Osmotin was also isolated by Singh et al (1987) by ammonium sulfate precipitation. Fraction I was collected as an 80% ammonium sulfate precipitate by centrifugation at 10,000 g at 4° C. after discarding the 40% precipitate. This was re-suspended in extraction buffer I and dialyzed for 20 h against five changes of 4 L distilled water at 44° C. Fraction II supernatant was precipitated with 80% ammonium sulfate, and a 10,000 g floating pad (because of the presence of detergent) was collected. This pad was suspended in extraction buffer 1 and dialyzed separately as was done for fraction I. After dialysis, fraction I was centrifuged separately at 10,000 g for 10 min, and the clear supernatant was collected. Fraction II, containing a turbid precipitate, was dissolved by adding an equal volume of 2× fraction II resuspension buffer (40 mM potassium phosphate buffer [pH 6.0], 8 M urea, and 0.2% NP-40).

Fraction I and Fraction II were further purified on CM-Sephadex® A-25 and SCX-polyamide HPLC. to get Osmotin I and Osmotin II respectively, which were dialyzed against water and suspended in buffers to get single 26 kD protein shown by SDS-PAGE.

Sephadex® stands for a porous dextran gel used as molecular sieve wherein after loading on to gel of Sephadex®, molecular species having largest molecular size gets eluted first and the molecular species having least molecular size elutes last and rest of the molecular species elute in the order of their molecular sizes, larger ones eluting earlier than the smaller ones.

Osmotin II has been seen to be resistant to most of the proteolytic enzymes. Osmotin I is more susceptible, however, its N-terminal portion is found to be resistant to proteolytic enzymes. Osmotin II was hydrophobic and particulate in nature.

Singh et al (1987) have pointed out that there is no functional assay for Osmotin and hence identification is based only on homogeneous band of Osmotin in a SDS_PAGE electrophoresis at a position of about 26 kD.

U.S. Pat. No. 5,389,609 has pointed out that the Osmotins of Tobacco “show a large amino-acid sequence identity, as well as similarity with a 24 kD Osmotin-like protein from tomato (Lycopersicon esculentum), and to other proteins, including thaumatin from Thaumatococcus daniellii, pathogenesis-related protein S (PR-5) from tobacco, and a bifunctional maize trypsin/.alpha.-amylase inhibitor. Osmotin-like proteins, which are all serologically related and have a molecular weight corresponding to those of Osmotins from tobacco and tomato are described, inter alia, for millet, soybean, carrot (Daucus carota), cotton, potato (Solanum tuberosum) (Singh et al., (1987), P.N.A.S. USA 84, 739-743), alfalfa (Medicago sativa), and bean (Phaseolus) (King et al., (1986), supra)”. For a long time, it was presumed that the only relevance of Osmotins is that they have function in providing the plant with osmotic tolerance after exposure to a low water potential. However, U.S. Pat. No. 5,389,609 disclosed that this class of PR-Proteins have anti-fungal effect, they retard the growth of fungi. U.S. Pat. No. 5,389,609 has also disclosed a general method for obtaining an antifungal protein with activity against an Oomycete, which are described as “Osmotin protein”. U.S. Pat. No. 5,389,609 has observed that “The antifungal activity appeared heat-labile. The active component from tobacco was identified as a hydrophobic protein with a molecular weight of about 24 kD and a basic isoelectric point (p1) and was called AP20 (also referred to as AP24). After purification and determination of the amino-acid sequence of the N-terminus of AP20, this part appeared identical to the corresponding part of Osmotin, a protein known to occur in tobacco (Singh et al 1987)” “and which belongs to a group of proteins designated as osmotins, or alternatively as osmotin-like proteins”. It is pointed out in U.S. Pat. No. 5,389,609 that accumulation of Osmotin-like proteins seems to be dependent on the continuous presence of osmotic agents. Under the influence of (some) non-osmotic agents like cadmium chloride accumulation does not occur (King et al., (1986), Plant Mol. Biol. 7, 441-449)”. U.S. Pat. No. 5,389,609 also points out to the PR-Proteins (pathogenesis related proteins), a class of Osmotin-like proteins in same range of molecular weight which can be induced to be formed as plant response to threat from pathogens or stress related conditions. The method disclosed by U.S. Pat. No. 5,389,609 for recovering the antifungal protein from an extract fraction comprises inoculating leaves of tobacco (Nicotiana tabacum cv. Samsun NN) of 7 to 8 weeks old plants with tobacco mosaic virus (TMV), harvesting leaves after seven days, homogenizing at 40° C. in a buffer containing 2-mercapto-ethanol and active carbon, using a Waring blender, filtering the homogenate over cheese cloth, centrifuging the filtrate at 5,000 g, collecting the supernatant and centrifuging for 50 minutes at 22,000 g, desalting on a Sephadex G25 column (Pharmacia), equilibrated in 20 mM NaOAc pH 5.2. The fraction containing proteins was centrifuged for 45 minutes at 22,000 g. The supernatant was passed through a S-sepharose “fast flow” (Pharmacia) column which was equilibrated with 20 mM NaOAc pH 5.2, at a flow Speed of approximately 15 ml per minute. The unbound proteins were collected. The bound proteins were eluted using an increasing linear NaCl gradient (0 to 500 mM) in the above mentioned buffer, and a flow speed of 3 ml per minute; fractions of 4.2 ml were collected. Each second fraction was analysed by electrophoresis using a 12.5% polyacrylamide gel in the presence of sodium dodecyl sulphate (SDS), using molecular weight markers of (20-66 kD) as reference. A separate portion of the same fractions was tested for antifungal effect. From 400 g leaf-material 400 μg of antifungal protein can be isolated (95% purity) or 200 μg antifungal protein of 99% purity.

U.S. Pat. No. 5,521,153 discloses an isolated and substantially purified Zeamatin having a molecular weight of about 22 kilo Daltons under reducing conditions and an N-terminal amino acid sequence as disclosed in SEQ. ID No.:1, wherein said Zeamatin decreases the minimum inhibitory concentration (MIC) of nikkomycin by about 10 to about 100-fold against strains of Candida albicans in in-vitro assays. The Zeamatin is isolatable from corn meal protein extract by a purification procedure which comprises the following steps: (1) isolation of the corn protein fraction precipitated between 30%-55% saturated ammonium sulfate: (2) application of said 30%-55% saturated ammonium sulfate precipitated fraction to a column of cross-linked carboxymethyl dextran of dry bead size 40-125 micron, washing said column to remove unbound protein, eluting protein fractions from said column using a linear salt gradient and isolating a first eluted protein fraction which displays SAFP (synergistic antifungal proteins) activity; and (3) application of said first eluted protein fraction to a hydrophobic column such as a column of porous linked phenyl agarose with a wet bead diameter of 45-165 micron, eluting protein fractions from said column using a low salt concentration aqueous solution and isolation of a second eluted protein fraction which displays SAFP activity; said second eluted protein fraction comprising said zeamatin.

Thus, for a long time, Osmotins and proteins homologous to them were ascribed anti-fungal and anti-stress activity.

Narasimhan et al (2005) (Narasimhan M L, Coca M A, Jin J, Yamagichi T, Ito, Y, Kadowaki T., Kim K K, Pardo J M, Damsz B., Hasegawa P M, Yun D J, Bressan R A, Osmotin is a homolog of mammalian adiponectin and controls apoptosis in yeast through a homolog of mammalian adiponectin receptor. IN Mol Cell, 2005 Jan. 21; 17(2):171-80.) disclosed for the first time that Osmotin and adiponectin do not share sequence similarity, however, the beta barrel domain of both proteins can be overlapped, and osmotin, like adiponectin, activates AMP kinase in C2C2 myocytes via adiponectin receptors. This finding has given a highly important role to Osmotins than the roles ascribed to them till disclosure from Narasimhan et al. Osmotin or Osmotin-like proteins are now a potential replacement for Adiponectin, a protein hormone, the deficiency of which in a human being is associated with a cluster of lifestyle disorders including insulin resistance and related disorders further including Type II diabetes, hyperlipidemia, atherosclerosis, hypertension, heart disease and obesity. Osmotin has been seen to act as an adiponectin agonist in mammalian systems. Hence Osmotin and its homologues PR-Proteins would be useful for the development of therapeutic methods for treatment of mammalian disorders in which adiponectin receptor-mediated remedies are possible.

In fact, the discovery of Adiponectin itself and recognition of its deficiency being associated with Type II diabetes, hyperlipidemia, atherosclerosis, hypertension, heart disease and obesity had given rise to potential of its use for treatment of these diseases/disorders. However, getting natural compositions of adiponectin in enough quantity is inconceivable and its synthesis on large scale is too complicated.

With findings of Narsimhan et al, Osmotin for the first time attracted attention of researchers as Adiponectin agonist.

U.S. Pat. No. 7,803,854 has disclosed a method for treating a mammal suffering from disorder selected from the group consisting of type II diabetes, insulin resistance, hyperlipidemia, arteriosclerosis, and heart disease, said method comprising administering to the mammal an isolated PR-5 protein, wherein said type II diabetes, insulin resistance, hyperlipidemia, arteriosclerosis, or heart disease is treated. The PR-5 protein is specifically illustrated by Osmotin. PR-5 proteins that are not isolated and are associated with vegetables are specifically excluded from the scope of the claims of U.S. Pat. No. 7,803,854.

Citing Narasimhan et al (2005), Lele (2010) “R.D. Lele; Pro-insulin, C Peptide, Glucagon, Adiponectin, TNFa, AMPK: Neglected Players in Type 2 Diabetes Mellitus”, JAPI⋅January 2010⋅VOL. 58 pp 30-36) pointed out that the beneficial effects of a diet containing 400 g. of fruits and vegetable may partly be due to the Osmotin content apart from the anti-oxidants and high fiber low fat content. Vandit et al (2012) [V. R. Trivedi, M. R. Chorawala, G. B. Shah, ANTIATHEROSCLEROTIC ACTIVITY OF OSMOTIN, A ADIPONECTIN AGONIST IN ATHEROGENIC DIET INDUCED HYPERTRIGLYCERIDEMIA AND HYPERCHOLESTEROLEMIA IN WISTAR RATS; ARPB, 2012; Vol 2 (II)] have shown that both Osmotin-I and Osmotin-II produced by method of Singh et al (1987) from salt adapted tobacco cell suspensions (Nicotiana tabacum L) resulted in decrease in body weight, serum triglyceride, total serum cholesterol and decreased atherosclerotic lesion area. These effects were comparable to that produced by atorvastatin.

However, eating 400 grams green leafy vegetables or 400 gram fruits per day does not seem to be a feasible way of getting significantly enough Osmotin in diet. The way vegetables are cooked and eaten, there is reason to maintain that the release of Osmotin from leaves could not be expected to be more than 3-10% since Osmotin is a large molecule, being a protein. If smaller molecule like beta Carotene is taken as an indicator molecule, release of same, in an experiment of Lopez et al (2010) [(Armando Carrillo-Lopez, 1Elhadi M. Yahia and 1,2Gabriela K. Ramirez-Padilla. Bioconversion of Carotenoids in Five Fruits and Vegetables to Vitamin A Measured by Retinol Accumulation in Rat Livers. American Journal of Agricultural and Biological Sciences 5 (2): 215-221, 2010)] has shown that this release could be only about 3% to 10%. In fact, even without a rat feeding, the fact of low release of phytonutrients from green leafy vegetables can be verified from the fact that the usual way of cooking and eating Fenugreek green leafy vegetable gives a slight bitter state which is actually agreeable to most people who relish eating this vegetable. However, same vegetable, if pulped in a kitchen blender before or after cooking, gives very strong repulsive bitter taste because far greater proportion of bitter principle of Fenugreek gets released from cells due to mechanical pulping. Further, considering the volume, eating 400 gram green leafy vegetables every day is a difficult task. Hence, eating vegetable in meals is not feasible way of getting full potential benefit. Fruits do give better bioavailability of phytochemicals contained in them, as shown by Lopez et al. However, many of them are associated with a content of sugars/energy with them which is unacceptable for a section of people including diabetics. Hence, getting plant fractions free from undesired factors but containing significant quantity of bioavailable Osmotin, which is amenable to be standardized for their Osmotin content is critical for practicality of considering vegetables and/or fruits as feasible sources of Osmotin.

However, getting enough Osmotin or Osmotin-like proteins produced from its currently known sources is difficult to achieve by methods known in prior art since it requires giving pathogenic or abiotic stress to the cultured cells or plants and involves several steps of ammonium sulfate precipitation or centrifugation at 10,000 g or both followed by gradient elution on carboxymethyl dextran (CM Sephadex®) or SCX-polyamide HPLC. Yield of Osmotin-like proteins reported by following the above mentioned steps is reported by U.S. Pat. No. 5,389,609 is 400 μg of antifungal protein of 95% purity or 200 μg antifungal protein of 99% purity from 400 g leaf-material. Further, none of these methods can be conveniently adapted to large scale manufacture. Far higher yields of isolated Osmotin will be required to be achieved from the stressed plant material to make method of use of Osmotin as functional homologue of Adiponectin or as Adiponectin agonist viable for using in treatment of diseases and disorders that arise from deficiency of Adiponectin.

Simpler methods that are amenable to scale up are required for viability of considering Osmotin or Osmotin like proteins as ingredients that are Adiponectin agonist in pharmaceutical preparations, which can be used to treat diseases and disorders arising from Adiponectin deficiency. Prior art has disclosed that such diseases and disorders at least include type II diabetes, insulin resistance, hyperlipidemia, arteriosclerosis, and heart disease. This is, in view of prior art knowledge, a very difficult task since Osmotin or Osmotin-like proteins make a very tiny part of total protein content in plants and so far separation of Osmotin or Osmotin-like proteins from overwhelming excess of protein impurities could be achieved only by reliance on methods based on Narasimhan et al (1987) or a modification thereof, that involve several steps of ammonium sulfate precipitation or centrifugation at 10,000 g or both followed by gradient elution on carboxymethyl dextran (CM Sephadex) or SCX-polyamide H PLC; and that too at a meager yield of 400 μg of antifungal protein of 95% purity or 200 μg antifungal protein of 99% purity from 400 g leaf-material. The requirement of Osmotin yields, however, to make it a practical proposition for therapeutic applications is far more than current levels of 200-400 μg.

Savangikar & Savangikar (2014) (WO 2014/195975) have disclosed a method of using for anti-pathogenesis effect a composition comprising heat stable plant juice soluble plant proteins; the contents of this publication are incorporated in this specification in their entirely by this reference. The composition is standardized for a parameter of a property of the composition that is relatable to anti-pathogenesis property or to the content of the protein molecules having molecular weight of 26 kilo Daltons (KD) or less. Also disclosed is an anti-pathogenesis composition comprising heat stable plant juice soluble plant proteins, wherein the heat stable plant juice soluble plant proteins are obtained as juice solubles by subjecting plant or plant juice to heat treatment to precipitate proteins, overwhelming excess of which are heat coagulable, along with a group of phytochemicals which co-coagulate with the heat precipitable proteins in plants leaving behind other plant constituents carrying a small quantity of heat stable juice soluble proteins in the juice to yield juice solubles, and wherein the composition is standardized for a parameter of a property of the composition that is relatable to the anti-pathogenesis property or to the content of the protein molecules having molecular weight of 26 kilo Daltons (KD) or less. Also disclosed is a method/process of making a composition comprising heat stable plant juice soluble plant proteins the process comprising steps of: (a) heat precipitating a plant juice and separating the macromolecules including proteins that coagulate on heating from soluble constituents to get juice solubles containing heat stable plant juice soluble plant proteins, (b) quantifying the heat stable plant juice soluble plant proteins by a method that is relatable to the anti-pathogenesis property or to the content of the protein molecules having molecular weight of 26 kilo Daltons or less, (c) concentrating or diluting to reach the figure of the parameter of a property that is targeted/chosen as standard, and, further optionally adding additive ingredients selected from the group comprising pharmaceutical actives, pharmaceutical excipients, cosmaceutical actives, cosmaceutical excipients, nutraceutical actives, nutraceutical excipients, plant protection actives, food additives or other food ingredients. Also disclosed is a plant material composition not containing heat stable proteins; wherein the composition is made by pulping the plant material, removing juice from the solid residue, heating juice to remove coagulable protein and non-protein matter as a coagulum, removing the coagulum and thereafter removing the heat stable proteins from the resulting composition, optionally treating the same to get a further composition which comprises either non-protein hydrophilic plant juice solubles or non-protein lipophilic plant juice solubles or both together in same composition; and optionally additive ingredients selected from the group comprising pharmaceutical actives, pharmaceutical excipients, cosmaceutical actives, cosmaceutical excipients, nutraceutical actives, nutraceutical excipients, plant protection actives, food additives or other food ingredients.

Savangikar & Savangikar (2014) illustrated that the concentrated plant juice solubles were effective in replacing dose of three 500 mg tablets of Metformin per day in a Type-2 diabetic person for lowering post-prandinal blood sugar level. The post-prandinal sugar in blood serum, however, was still above 200 mg/dl on most of the days. Further, consuming two to three spoons of concentrated juice solubles two or three times a day present difficulties in practical adaptation of the composition in regular use because concentrated juice solubles syrup is extremely unpalatable being high in bitter and astringent taste, and becomes difficult in carrying, dispensing and accurate dosing. Thus, improvement was needed. Further, it was seen that the composition was not storage stable, which was indicated by the fact that the efficacy decreased progressively in storage.

### SUMMARY OF THE INVENTION

This invention comprises a method of using for anti-pathogenesis effect an improved composition comprising heat stable plant juice soluble plant proteins; the improvement comprising that the composition comprising heat stable plant juice soluble plant protein is essentially a water insoluble concentrated mass free from components more soluble in the plant juice, and enriched in heat stable proteins of the plant juice.

This invention also comprises an improved composition of heat stable plant juice soluble plant proteins; wherein the improvement comprising in (a) essentially water insoluble property of the concentrate mass, (b) the concentrated mass being essentially free from components more soluble in the plant juice, and (c) being enriched in heat stable proteins in comparison with composition of plant juice solubles; wherein the composition comprising heat stable plant juice soluble plant protein is essentially a water insoluble concentrated mass free from components more soluble in the plant juice, and enriched in heat stable proteins of the plant juice.

The composition of this invention also comprises dosage forms made from the composition of claim 2.

In one aspect of this invention, the composition is standardized for a parameter of a property of the composition that is relatable to anti-pathogenesis property or to the content of the protein molecules having molecular weight of 26 kilo Daltons (kD) or less.

In another aspect the anti-pathogenesis effect of the above described composition comprises an anti-microbial effect on living beings including microbes or as an Adiponectin agonist effect in mammals.

The method of using the composition of this invention comprises the Adiponectin agonist effect in mammals further comprising reducing the risk of one or more of a disorder selected from Type II diabetes, hyperlipidemia, atherosclerosis, hypertension, heart disease and obesity.

The method of using the composition of this invention described above wherein the anti-microbial effect comprises reducing the risk of or treating the microbial infections in plants and animals, with or without synergistic interaction with other antimicrobial agents.

This invention also comprises an improved antipathogenesis composition comprising heat stable plant juice soluble plant proteins, wherein the heat stable plant juice soluble plant proteins are obtained as juice solubles by subjecting plant or plant juice to heat treatment to precipitate proteins, overwhelming excess of which are heat coagulable, along with a group of phytochemicals which co-coagulate with the heat precipitable proteins in plants leaving behind other plant constituents carrying a small quantity of heat stable juice soluble proteins in the juice to yield juice solubles, and wherein the composition is standardized for a parameter of a property of the composition that is relatable to the anti-pathogenesis property or to the content of the protein molecules having molecular weight of 26 kilo Daltons (kD) or less; the improvement comprising that the composition comprising heat stable plant juice soluble plant protein is essentially a water insoluble concentrated mass free from components more soluble in the plant juice, and enriched in heat stable proteins of the plant juice.

The composition of this invention may be standardized for a parameter of the property of the composition is determined in terms of one or more of the analytical parameters selected from the group comprising: (a) dry matter percentage of the composition, (b) nitrogen percent of dry matter of the composition, (c) content in terms of casein or gallic acid, (d) by molecular weight determination, or by (e) Forurier Transform Infra Red spectra.

This invention comprises composition of the heat stable plant juice soluble plant proteins which are used with or without pharmaceutical actives, pharmaceutical excipients, cosmaceutical actives, cosmaceutical excipients, nutraceutical actives, nutraceutical excipients, plant protection actives, food additives or other food ingredients.

In another aspect, this invention comprises a method/process of making a composition comprising heat stable plant juice soluble plant proteins the process comprising steps of:
a. heat precipitating a plant juice and separating the macromolecules including proteins that coagulate on heating from soluble constituents to get juice solubles containing heat stable plant juice soluble plant proteins,
b. quantifying the heat stable plant juice soluble plant proteins by a method that is relatable to the anti-pathogenesis property or to the content of the protein molecules having molecular weight of 26 kilo Daltons or less,
c. concentrating or diluting to reach the figure of the parameter of a property that is targeted/chosen as standard, and,
d. further optionally adding additive ingredients selected from the group comprising pharmaceutical actives, pharmaceutical excipients, cosmaceutical actives, cosmaceutical excipients, nutraceutical actives, nutraceutical excipients, plant protection actives, food additives or other food ingredients;
   wherein,
   the composition comprising heat stable plant juice soluble plant protein is essentially a water insoluble concentrated mass free from components more soluble in the plant juice, and enriched in heat stable proteins of the plant juice.

Composition of this invention is also embodied in the dosage forms comprising concentrated syrup, chewable or orally dissolving tablets, granules, powder, immediate delivery tablets, intermediate delivery tablets, controlled delivery tablets, capsules, orally dissolvable films with or without microemulsion of the improved composition.

This invention comprises a composition essentially characterized by Infra-red absorption spectrum comprising a peak at around 3365 wavelength having % transmittance of between 35-60, a peak at around wavelength 2975 having % transmittance of between 60-80, a peak at around wavelength 1612 having % transmittance of between 60-80, a peak at around wavelength 1306 having % transmittance of between 65-70, a peak at around wavelength 1280 having % transmittance of between 70-90, a peak at around wavelength 1080 having % transmittance of between 20-40, a peak at around wavelength 887 having % transmittance of between 70-98 and a peak at around wavelength 825 having % transmittance of between 85-98; wherein the composition comprises heat stable plant juice soluble plant protein that is essentially a water insoluble concentrated mass free from components more soluble in the plant juice, and enriched in heat stable proteins of the plant juice.

### DETAILED DESCRIPTION OF THE INVENTION

Brief description of figures and legends:

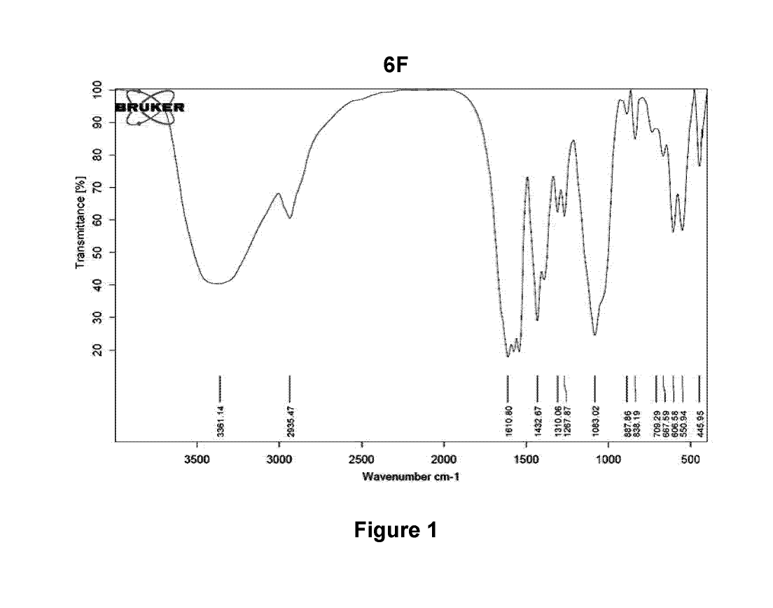

FIG. 1: Fourier Transform Infra Red (FT-IR) spectrum of improved composition 6F.

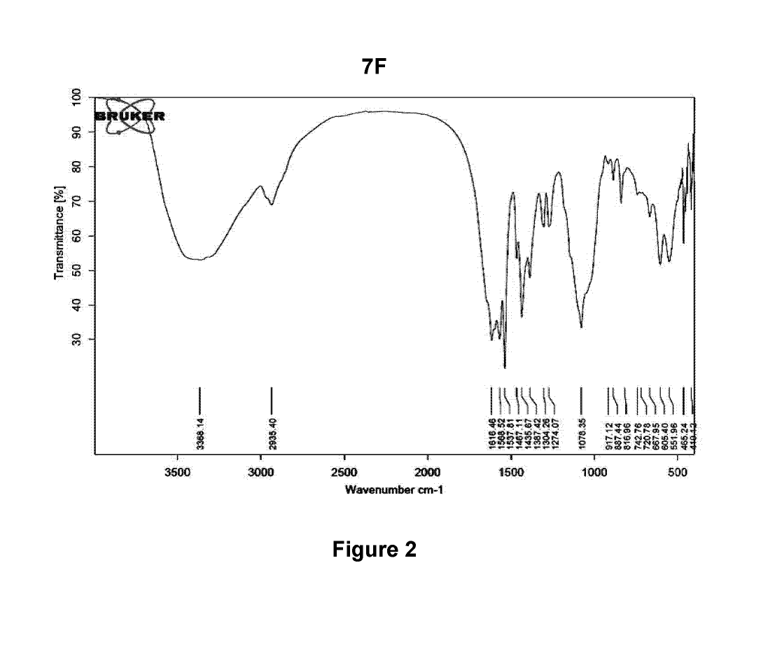

FIG. 2: Fourier Transform Infra Red (FT-IR) spectrum of improved composition 7F.

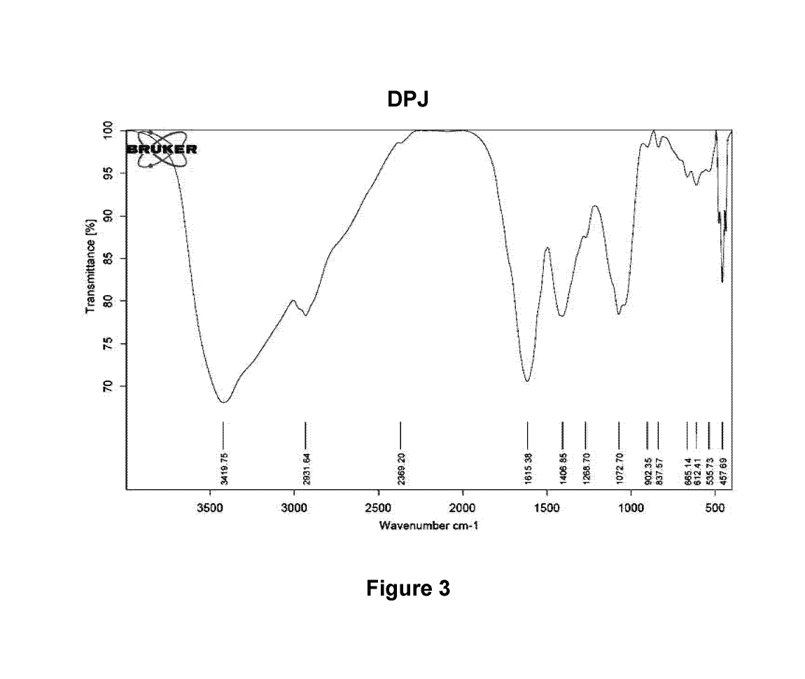

FIG. 3: Fourier Transform Infra Red (FT-IR) spectrum of the prior art composition of Savangikar and Savangikar (2014) DPJ.

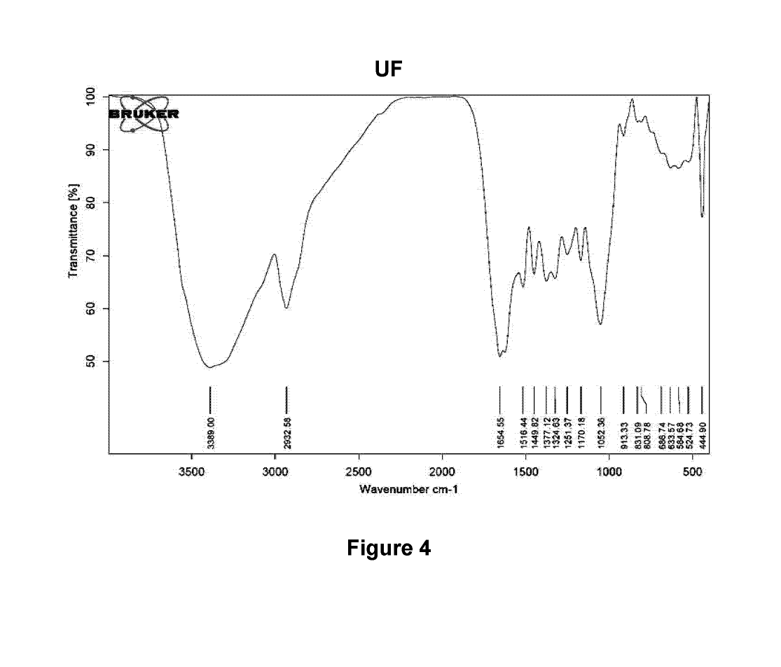

FIG. 4: Fourier Transform Infra Red (FT-IR) spectrum of improved composition UF.

The composition of Savangikar & Savangikar (2014) (FIG. 3), which has shown anti-pathogenesis property by reducing the blood sugar levels even lower than the blood sugar levels obtained by using Metformin, was concentrated leaf juice soluble which contained all the soluble ingredients of leaf juice in addition to the “heat stable plant juice soluble plant proteins”. On one day, urine sugar level remained zero throughout the day when the concentrated whole juice solubles (FIG. 1) were consumed in addition to Metformin, showing additional blood sugar reducing effect in view of the fact that without the juice soluble only Metformin showed almost four times higher blood sugar level (table 1, page 3 of Savangikar & Savangikar, 2014). Thus, it was concluded that the whole concentrated leaf juice solubles did retain their activity although the leaf juice was given a heat treatment to coagulate and to remove high molecular weight proteins from the juice to leave behind only juice solubles containing heat stable proteins and other juice soluble ingredients. Thus, in the context of prior art, it was observed that isolation of PR_5 proteins as 95-95% pure isolate was no more required to augment reasonable Adiponectin agonist activity, and yet a composition containing efficacious level of Adiponectin agonist proteins was made for the first time and validated for use as anti-pathogenesis composition.

However, the observation in Savangikar & Savangikar (2014) was that two year old juice syrup did not exhibit any Adiponectin agonist activity and the activity was seen as soon as freshly prepared juice soluble syrup was administered, suggesting that the activity of the heat stable juice soluble proteins in the concentrated juice soluble was not stable. This was seen to be applicable for the freshly prepared juice soluble syrup also since observations continued after the reported period in Savangikar and Savangikar (2014) did show slow decrease in the activity of the juice solubles syrup and after the syrup was exhausted, Metformin was started again, this time 500 mg each at breakfast, lunch and before going to sleep. At this dosage regime, it was observed, based on regular urine testing by Benedict's reagent on two hours post-prandinal as well as pooled urine samples of each period between two food intake events (morning fruits and occasional sweet snack, breakfast, lunch, evening dinner; and pooled urine sample of night bed time small snack to next day morning, that blood sugar level was always below renal threshold of 180 mg/dl after dinner in the evening and between the small night snack to next day morning pooled urine sample.

Savangikar and Savangikar (2014) opted to use the concentrated juice soluble for anti-pathogenesis effect wherein the requirement for activity will be minimal, i.e. to be able to keep activity of the heat stable juice soluble proteins intact even during concentration by boiling. This was accomplished as shown by the blood sugar lowering efficacy of the freshly prepared concentrated juice soluble. However, in view of the problems for the concentrated syrup of leaf juice soluble comprising unpalatability, slow decline in storage stability and difficulty to convert into a convenient dosage form, further improvements were necessary.

For the desired improvement, it seemed necessary to improve the relative content of the heat stable juice soluble proteins by removing non-protein soluble constituents from the concentrated juice of Savangikar and Savangikar (2014) so that the same has Improved palatability, gets further concentrated to provide a concentrate of heat stable juice soluble proteins, the processing conditions used should not have any adverse effect on the anti-pathogenesis activity of heat stable juice soluble proteins and the cost of production should also be within practical limits. Such a concentrate composition of heat stable juice soluble proteins shall be amenable to make convenient dosage compositions.

In prior art described above [Singh et al (1987), U.S. Pat. Nos. 5,389,609, 5,521,153] for making active preparations of PR-5 protein with anti-pathogenesis activity, methods used involved extremely mild conditions of processing involving gel filtration over columns, affinity chromatography over adsorbents in columns by elution ingredients and getting very small yields. In fact this is typical of any composition of biologically active protein molecule of any type. On this background, it is a surprising embodiment of this invention that the composition of heat stable plant juice soluble plant proteins of this invention having anti-pathogenesis activity is essentially a water insoluble composition. The finding that the water insoluble composition containing the heat stable plant juice soluble plant proteins was also effective as anti-pathogenesis use, simpler methods of separation and to concentrate compositions enriched in these proteins and substantially free from soluble components of non-protein juice soluble could be prepared.

Thus, in one aspect of this invention, this invention comprises a method of using for anti-pathogenesis effect an improved composition comprising heat stable plant juice soluble plant proteins; the improvement comprising that the composition comprising heat stable plant juice soluble plant protein is a water insoluble concentrated mass essentially from components more soluble in the plant juice, and enriched in heat stable proteins of the plant juice.

In another aspect, this invention comprises an improved composition of heat stable plant juice soluble plant proteins; wherein the improvement comprising in (a) essentially water insoluble property of the concentrate mass, (b) the concentrated mass being essentially free from components more soluble in the plant juice, and (c) being enriched in heat stable proteins in comparison with composition of plant juice solubles.

In another aspect, this invention comprises compositions of dosage forms made from the above described improved composition. In a further aspect, the dosage forms include concentrated syrup, chewable or orally dissolving tablets, granules, powder, immediate delivery tablets, intermediate delivery tablets, controlled delivery tablets, capsules, and orally dissolvable films with or without microemulsion of the above described improved composition of this invention.

This invention also comprises a method/process of making the composition of this invention by step of adding ascorbic acid to the juice solubles prepared by coagulating juice of green leafy vegetation in a quantity that would prevent oxidative reactions, further followed by one or more of the steps comprising: (a), standing the same for a period of time to allow insolubilized solids to appear and (i) allowing the solids to settle at the bottom, decanting the supernatant or (ii) centrifugally separating the insolubilized solids before or after sedimentation from the juice solubles, or (b) concentrating the juice solubles by ultrafiltration or boiling off and further decanting or filtering off or centrifuging the insolubles off from the retaintate.

It has been found that Fourier Transform Infra Red (FT-IR) spectrum of the improved compositions (UF, 6F and 7F described in examples below) of this invention prepared by above mentioned variations of the method is essentially same confirming thereby that the composition/product formed by above described variations of making the composition of this invention result in same product and the composition is also reproducible in its nature and structure. It has been observed that all of them result in lowering of post-prandinal blood sugar when consumed as a supplement with meals and could totally replace Metformin dose of 1 500 mg tablet three times a day with breakfast, lunch and bed time snack. It was also observed that High Density Lipoprotein which was very low, improved up to normal level, Very Low Density Lipoprotein (VLDL), Low Density Lipoprotein and triglycerides reduced and came in normal range. Hypertension also returned to normal level. This indicates that the composition of this invention does have Adiponectin Agonist properties, which can be contributed reasonably only by PR-5 proteins i.e. Osmotin or Osmotin-like proteins.

Analysis of the compositions of this invention (6F and 7F) showed that they have 9% protein. When suspensed in water, a lot of their portion settles at bottom, but some golden yellow matter makes the supernatant clear golden yellow in colour. Thus, it seems that the treatment that insolubilizes a portion of the juice solubles comprises protein as well as non-protein matter, of which, when enough water is available, the protein part again gets solubilized and it is this part of the composition that provides the anti-pathogenesis activity. Hence, it is possible that this soluble component may also be amenable to concentration without damage and help in getting further concentrated composition, which and dosage forms derived form it is/are also included within the scope of this invention.

Anti-pathogenesis activity of PR-5 protein family is well known and established in pant science. Hence, the composition of this invention would also be useful for anti-microbial purposes, either on its own or with other anti-microbials with a additive or synergistic effect; and such applications of the composition of this invention are also included within the scope of this invention.

Syrups of the improved composition of this invention were used in dropping bottles with concentration of the dry matter calibrated to dispense known quantity of the improved composition per drop or per spoon. Immediate release tablets of 40 and 80 mg of the improved composition were made and consumed four times a day and slow release tablet of 320 mg was consumed two tablets per day at 12 hours intervals. Whether as syrup or as a tablet, the dosage of the composition of this invention was taken four times a day: one immediately after getting up with morning fruit, and one each at breakfast, lunch and night-time snack before sleeping. It was observed that when given as an add-on treatment to Metformin consuming patients having post-prandinal sugar level dropped down sharply immediately on almost the same day the dosage of this invention was administered. The Metformin could be phased out with stepwise reduction in the dosage within a span of 30 days; and even after continuing exclusively on the composition of the instant invention without Metformin for one year, the blood sugar levels slowly increased again, but never crossed the blood sugar levels noted before start of the dosages of the composition of this invention as an add-on treatment. Thus, it can be concluded that the composition of this invention is capable of at least replacing Metformin; and provides additional effect than Metformin in that it also normalizes lipid profile, triglycerides and brings down the dosage of hypertension drug.

It is also observed in the instant invention that in syrup form, no difference in efficacy was noted when the dose was increased from 80 mg/dose to above stepwise up to 320 mg/dose. No improvement was noted also with slow release tablet of 320 mg/tablet. However, substantial improvement was noted when immediate release tablet of 40 mg/tablet was consumed. This observation was also surprising, being contrary to the expectation that drying of the active protein for tablet making would decrease the activity and efficacy of the composition of this invention.

Invention of the water insoluble form of heat stable plant juice soluble plant proteins as an active composition for providing anti-pathogenesis effect provided a technical advance. This product could be easily made and recovered from the juice solubles by using inexpensive equipment and by scalable economically viable process requiring no costly chemicals/adsorbents and the active composition could be easily made into tablets which gave best efficacy. It is also feasible to envisage its application for plant protection compositions on account of its remarkable stability with no requirement of buffers etc. As a matter of abundant precaution, the slurry of the insoluble composition recovered from the juice solubles was adjusted to pH 6.5 by adding Di Potassium Hydrogen Phosphate and Potassium dihydrogen phosphate to make it 20 mM with respect to the potassium buffer pH 6.5 and potassium sorbate and sodium benzoate were added 50:50 to make 300 ppm with respect to the volume of the slurry for their preservative action. However, it has not been verified to which extent the buffer, pH adjustment and the preservatives are essential in the slurry.

“Juice solubles” is understood as juice derived from plants which contains in it heat stable proteins and is free from heat coagulable proteins.

The “juice solubles” are defined as an aqueous liquid composition made from plants from which heat coagulable components have been removed and the composition comprises plant derived ingredients that do not coagulate by heat. In the illustration provided in this specification, the “Juice solubles” are derived from the juice obtained from pulped green leafy vegetation/vegetable by pressing or centrifugation and the green juice obtained thus is heated to 80° C. or above to coagulate heat coagulable proteins and removing the coagulated proteins and co-coagulated nutrients from the heated brown juice. This composition can also be obtained from pulp of the green leafy vegetation by subjecting it to heating up to 80° C. or above to coagulate heat coagulable proteins and removing the juice insoluble constituents from the mass to get brown liquid juice containing solubles.

Importance of this invention emerges from the central role that Adiponectin plays in maintaining health of human being as a protein hormone, the deficiency of which in a human being is associated with a cluster of lifestyle disorders including insulin resistance and related disorders further including Type II diabetes, hyperlipidemia, atherosclerosis, hypertension, heart disease and obesity. Because Adiponectin is a protein molecule which could not be made synthetically so far, the disorders that arise on account of its deficiency are required to be treated with as many medicines as the number of disorders it throws up as separate diseases, each medicine having its own side effects and its own drug interactions that adversely affect quality of life. Multiplicity of medicines required to be consumed per day is by itself an additional risk factor for patient compliance at all ages. Lifestyle changes to reduce the risk of lifestyle diseases is seen to have a limited utility. Availability of an efficacious and affordable Adiponectin Agonist shall radically change the nature of the course available for managing the lifestyle disorders. It would provide a single composition that would substantially reduce the risk of ALL lifestyle diseases and disorders listed above that result from Adiponectin deficiency, may eliminate the need of or substantially reduce the dose of multiple chemical medicines needed to treat and manage lifestyle diseases and disorders and would be the most logical first line response to lifestyle disorder/s and the multiple chemical medicines would be needed only to the extent to which the disease or disorder still persists even after administration of the Adiponectin agonist. The Adiponectin Agonist concentrate of this invention is illustrated herein as an extract made from green leafy vegetable Methi (Fenugreek, Trigonella foenum-graecum L.). However, it is known that Osmotin and Osmotin-like proteins are ubiquitous in all plant species (U.S. Pat. No. 7,803,854; column 9 lines 38-40), hence, the composition of this invention can be made from green leafy vegetation of plant species any other than fenugreek.

Below is described a examples which illustrate compositions and methods of making the compositions comprising Osmotins or Osmotin-like proteins which are also known as PR-5 proteins, which would satisfy the above described long unmet need. A person skilled in the art would immediately realize from ubiquitous nature of this family of proteins that although the illustration is made by using fenugreek green leafy vegetable, the scope of this invention covers within its scope all plant species from which fractions of heat soluble proteins can be obtained and separated as concentrates by adapting methods described herein.

The “composition comprising heat stable plant juice soluble plant proteins” illustrated to have anti-pathogenesis activity by Savangikar and Savangikar (2014), the syrup of concentrated juice soluble, is a different composition of matter than the “improved composition of heat stable plant juice soluble plant proteins; wherein the improvement comprising in (a) essentially water insoluble property of the concentrate mass, (b) the concentrated mass being essentially free from components more soluble in the plant juice, and (c) being enriched in heat stable proteins in comparison with composition of plant juice solubles” of this invention. This has reflected very much in the distinctly different FT-IR (Fourier Transform Infra Red) spectra of dried off juice soluble (the “composition comprising “heat stable plant juice soluble plant proteins” of Savangikar & Savangikar (2014) (FIG. 1) and the improved composition of this invention i.e. the improved composition of heat stable plant juice soluble plant proteins; wherein the improvement comprising in (a) essentially water insoluble property of the concentrate mass, (b) the concentrated mass being essentially free from components more soluble in the plant juice, and (c) being enriched in heat stable proteins in comparison with composition of plant juice solubles (FIGS. 2, 3 and 4).

Following are non-limiting examples of working of the invention. Variations of the same that are obvious and equivalents would be readily apparent to a person skilled in the art and the same are also included within the scope of this invention.

### EXAMPLES

### Example 1

Preparation of a Syrup of Water Insoluble Concentrated Composition Comprising Heat Stable Plant Juice Soluble Plant Protein Free From Components More Soluble in the Plant Juice, and Enriched in Heat Stable Proteins of the Plant Juice:

In one embodiment of further improved heat stable plant juice soluble plant proteins in a method for use for antipathogenesis, the juice solubles were prepared by pulping the shoots of the green leafy vegetable of Methi (Trigonella foenum-graecum L) by adding ascorbic acid solution containing about 380 mg ascorbic acid per kg vegetable and expressing juice from the pulp obtained. The juice was coagulated by heating at 80-100 degrees celcius and filtered through a cloth to get a green coagulum on the cloth and a filtrate, juice solubles, that are collected below, which is named for the purpose of this specification as “juice solubles”. To the filtrate/, the “juice soluble”, additional ascorbic acid was added to such an extent that brown color changed to golden yellow. In general, this amounted to about 200 grams additional ascorbic acid per 150 liter of the juice soluble. This quantity of ascorbic acid can change from batch to batch of the vegetable since it shall depend upon the phenols content of the same. The purpose is to avoid oxidation of phenols and also to avoid protein-phenol interaction, and any other oxidative reactions. The volume of the above mentioned ascorbic acid solution at the time of pulping was adjusted such that the weight of the juice solubles at the stage of adding additional ascorbic acid is about the same as the weight of the vegetable pulped.

The juice solubles were processed further in following different ways:
a) In one embodiment, the juice solubles were passed through an ultra-filtration membrane of 1 KD cut-off to separate less than 1 KD molecular weight species in the filtrate from rest of the juice solubles to get the 26 KD or less fraction of proteins in juice soluble in the about six times concentrated retained fraction. In the illustrative experiment, 1 KD cut off membrane was used because that one was available. It is also possible to consider using two cut off membranes, one of 20 KD and other of 26 KD cut off membrane so as to get a fraction of retentate; fraction that was retained on the ultrafiltration membrane and could not pass off through membrane in the permeated/filtered off fraction. The retentate was 6 times concentrated fraction (Improved Composition labeled as: IC 1). The retentate contained high molecular weight proteins and phenols that were contained in the juice solubles and showed sharp reduction in post-prandinal glucose when administered as an add-on to the Metformin therapy of three 500 mg tablets per day, one with each major meal (breakfast, lunch and dinner). From the retentate, the heat stable protein-containing concentrated fraction made from the juice solubles was obtained as a sediment by subjecting the same to centrifugation at a such revolutions per minute for a period as to be sufficient to separate the precipitate from clear supernatant. In the illustrative experiment, 5000 revolutions per minute for 5 minutes was used for separation of the precipitate as a sediment. The sediment would comprise the 26 KD and below molecular weight proteins of the interest, and the same was re-suspended in water to an extent which was a 16.6 times reduction in total volume with respect to the volume of juice from which the sediment was obtained; i.e. 16.6 times concentration of the high molecular weight species in the juice solubles including the proteins of interest. When filled up in a bottle containing a nozzle that provides droplets of the contents when inverted and pressed, it was seen that 25 drops make up for one ml of the composition. Thus, dispensing measured quantity of the active ingredient from this device was also convenient. It is also very much possible to re-suspend in much smaller quantity of water so that lesser number of drops would be required to dispense same quantity of proteins of interest in less number of drops more conveniently. As a matter of precaution, the water used for re-suspending the sediment after centrifugation is added with 0.5 g ascorbic acid per liter of water. The final volume of re-suspended sediment was added with K₂HPO₄ and KH₂PO₄ to make the entire volume 20 mM with respect to the resulting phosphate buffer pH 6.5. The resulting composition was a buff colored suspension that needed to be shaken before use as drops dispensed from the dropping bottle (Improved composition: IC 2).
b) In another embodiment, the juice solubles were concentrated to about 4 times or more times by heating to boiling and kept standing to let a precipitate to settle. The precipitate collected at the base of the holding container is collected by decanting most of the supernatant, and last portions of supernatant are removed by centrifugation or washing three times by resuspending in water to which 500 mg ascorbic acid per liter of water was added, allowed to stand, settle and the supernatant was discarded and the slurry of the precipitate was collected (two separately prepared batches of these improved compositions were labeled as 6F and 7F). The precipitate, by reasoning, would contain the proteins that are soluble in the juice solubles sparingly but get precipitated when concentrated, and would be of a molecular weight 26 KD or less. For getting syrup for consumption, a part of the slurry was suspended in water containing K₂HPO₄ and KH₂PO₄ to make the volume 20 mM with respect to phosphate buffer of pH 6.5 to contain desired dry matter content per spoon. For getting solids for making tablet or for chemical composition analysis, the slurry was spread over a polythene sheet and dried under a fan at room temperature of about 25° C. (from batches labeled as 6F and 7F).
c) In a third embodiment, the juice solubles were centrifuged through a decanter centrifuge at 16000 g to get a sediment containing the high molecular weight components, more than 10,000 KD, that were soluble in the juice solubles, and juice solubles substantially free from high molecular weight components. The sediment (the improved composition labeled as UF) was about 45% in dry matter and on dry matter basis the yield of this component was about 0.15-0.2% of the weight of the fresh green vegetation of Methi. This was resuspended in water to which KH₂PO₄ and K₂HPO₄ were added to make the entire volume 20 mM with respect to a pH 6.5 phosphate buffer and to make desired concentration of the dry matter of the sedimented solids per spoon of 4.5 ml.
d) Tablets were prepared from the dried powders of the improved compositions labeled as 6F and 7F They were of following compositions:
   a. Immediate release tablets of 40 mg and 80 mg active ingredient per tablet: These tablets contained, per tablet, slurry of 7F taken to provide an equivalent of 40 mg or 80 mg per tablet as the case may be, Microcrystalline Cellulose 93 mg, Povidone™ (polyvinylpyrrolidone having a viscosity of 44000-54000 cps) 30 mg, xanthan gum 0.5 mg, and whey protein concentrate to make total ingredients to 500 mg. Talc was used as glident.
   b. Slow release tablet: These tablets contained, per tablet, slurry of 7F taken to provide an equivalent of 320 mg, Carbopol™ (a high molecular weight polymer of acrylic acid crosslinked with allyl ethers of pentaerythritol) 100 mg, Microcrystalline Cellulose 93 mg, Povidone 30 mg and whey protein concentrate to make total ingredients to 500 mg. Talc was used as glident.
The slurry of 7F was mixed with whey protein concentrate as diluents/filler with addition of water as required to make a paste. For slow release tablets, carbopol™ was also added to the paste. The paste was mixed in domestic waring blender/mixer to make a well mixed smooth paste. This was dried as a thin film over a polythene sheet under a fan at room temperature of about 25° C. and powdered in domestic mixer.
The dried powdered mix of the active ingredient and whey protein concentrate was sifted through 40 mesh sieve and further mixed thoroughly with Microcrystalline Cellulose and passed through 40 mesh sieve. The mixture was granulated with solution of Povidone. For slow release tablets of 320 mg, the granulated mix could be pressed into 1 gram tablets without any difficulty. For Immediate Release tablets of 40 and 80 mg, the tablets did not get sufficient hardness. Hence, the granulated mix was further added with xanthan gum solution made in water, mixed thoroughly by brief whipping in a mixer/grinder ensuring that the granules did not break and only the xanthan gum solution was uniformly applied over the granules. The granules were dried under a fan at room temperature until the surface of the granules was dry and did not stick up to each other, and the tablets were made.

### Example 2

Fourier Transform Infra Red (FT-IR) spectra of the improved compositions 6F (FIG. 1), 7F (FIG. 2), UF (FIG. 4) have been provided in comparison with the FT-IR spectrum of the concentrated juice solubles (DPJ—FIG. 3) of Savangikar and Savangikar (2014).

### Example 3

Effect of Improved Composition of Heat Stable Plant Juice Soluble Plant Proteins on Type-II Diabetic Persons:

This case study comprises self monitored and self reported Urine sugar observations were kept in a case study of a 65 year old 5 feet 6 inches tall type-II diabetic male human subject (Subject 1) weighing 65 kg, who is also an expert in handling laboratory techniques required to make urine sugar determinations by using Benedict's reagent, and has office attached to residence comprising a sedentary table work job. This is the same person on whom the data of efficacy of the anti-pathogenesis composition was illustrated by Savangikar and Savangikar (2014). The urine sugar was monitored by estimating on urine samples practically every day between getting up and breakfast, breakfast to lunch, lunch to evening dinner, between evening dinner and early night bed time snack at about 21.15 hours, between early night bed time snack and late night time and at the wake up time. The urine sugar was tested by qualitative Benedict's reagent at above samples between February 2016 to March 2017, except when travelling out of the station.

After the termination of the experimental period reported in Savangikar and Savangikar (2014), the Subject 1 continued to consume the then freshly prepared juice concentrated syrup until it exhausted and then returned to a dosage of one 500 mg Metformin tablet each at breakfast, Lunch and early night bed time snack. The diet and exercise routine was as follows: getting up from bed at about 5.00 hrs, eating one medium size banana weighing about 90-100 gram with peel (or a sweet laddu, i.e. a ball of gram flour fried in ghee and added with about 20 g sugar per laddu when banana was not available, taking a pill of Amlodipine, getting to table-work in residential office, tea at about 7.00 hrs without sugar, breakfast at 8.45 hrs. The breakfast consisted of two chapattis made from 30 gram whole wheat flour each and 100 ml cow milk and one boiled egg. This was immediately followed by an exercise known as “Suryanamaskar” six times, which comprises for each time a reiterative series of 12 positions that involve forward bending, backward bending, stretching to legs, balancing prostrate body on the two arms etc. Each position was maintained until four breaths. This was followed by small sideways bending exercise to the spinal cord on both sides until counting 15 breaths each side, flipping both arms sideways up to the head 30 times and raising each leg up up to the waist with a force 30 times, This was followed by start, 30 minutes after the breakfast time, for a 30 minutes morning walk on jogging track covering 2 kilometers. This was followed by table-work until lunch was taken at 13.15 hrs when lunch was consumed consisting of two chappatis, dahl 18 g, one boiled egg and one portion of vegetable. This was followed by table-work until 17.00 hrs when tea or coffee without sugar is consumed, again followed by table-work until 18 hrs when early dinner was consumed consisting of consisting of two chappatis, dahl 18 g and one portion of vegetable. This was followed by six “Suryanamaskars” and evening walk 30 minutes after dinner for 2 kilometers traversed in 30 minutes. This was again followed by table-work up to 21.15 when one and a half chapatti, 18 gram dahl, one egg without egg yellow were consumed and going to bed at about 22.00 hrs.

In this routine, it was observed that on most of the days, whether on Metformin or on syrup of the improved composition of this invention, urine sugar level was always zero at the time of morning (fasting) sample, for interval from getting up to breakfast, evening dinner to early night bed time snack and early night bed time snack to late night urine sample. The urine sample from breakfast to lunch and from lunch to evening dinner carried sugar.

It was surprising to find that, when with Prior to the start of administering the improved compositions of this invention, the Subject 1 had returned to the dose regime of three 500 mg tablets of Metformin, one each at the time of breakfast, lunch and bed time snack. The blood picture was 138 mg/dl fasting and 224 mg/dl on this dosage regime which was being followed for a long time. Thus, for above dose regime, this blood picture was a stabilized status.

It was a surprise when 50 ml of the improved composition labeled as IC1, which corresponded to dry matter content of about 80 mg of the 5 ml of 6 times concentrated ultra-filtered juice soluble of Methi (Trigonella foenum-graecum L) (Fenugreek) green leafy vegetable, was administered after food as an add-on treatment to Metformin, from the same afternoon, the urine sugar tested negative indicating that post-prandinal sugar level had came down below the renal threshold i.e. 180 mg/liter. The IC 1 was highly palatable since it had no bitter and astringent taste and had no disagreeable flavor. This treatment was repeated for lunch and dinner too, and the urine sugar test after lunch and the pooled urine samples until next day morning were also negative. Even with Metformin, never before at any time in few years prior to this observation was the urine sugar after breakfast and lunch negative. The rapidity with which the action was seen was also surprising. There was apparently no need to build up high concentration of the active ingredient in blood. Pooled up urine of post-breakfast, post-lunch as well as post-dinner at late evening and after small bed time snacks at about 21.15 hours in the night and up to morning The negative test for urine following the single administration of the 5 ml dose of the 6 times concentrated juice soluble continued for three consecutive days. On fourth day, the blood sugar levels were checked in a pathology laboratory and were 130.4 mg/dl for fasting and 177.1 for post-prandinal stage

With unexpectedly favorable results of the IC-1 composition, to explore convenience of administering lesser volume, the six times ultra-filtration-concentrated juice solubles fraction made through 1 KD filter was centrifuged at 7000 rpm for about 2.5 minutes, the collected precipitate was re-suspended in water containing 0.5% ascorbic acid solution in water to correspond to 16.6 times concentration with respect to the volume of concentrated juice soluble. Thus, the concentration achieved for this fraction starting from the juice soluble from each one kg vegetable was 6×16.6=100 times. To this concentrated solution was added K₂HPO₄ and KH₂PO₄ to make the liquid phase 20 mM with respect to the phosphate buffer and 0.3 ml dose of this composition was decided to be consumed in place of 5 ml of dose prior to concentration. This dose also kept on maintaining the test of urine sugar to negative, thereby confirming that the blood glucose level is at least lesser than renal threshold throughout 24 hours. Thus, this is a simplest known process known so far to get a composition of green leafy vegetables comprising heat stable plant juice soluble plant proteins below 26 KD, the Osmotin or Osmotin like proteins, in a small, concentrated, easily useable volume having an anti-pathogenesis effect/efficacy illustrated in the context of lowering post-prandinal blood sugar level. It was also surprising to note that this fraction, which seems to be a precipitate, would ordinarily be understood as an inactive protein composition, kept its activity/efficacy intact without any loss even after precipitation and was active as an agonist of Adiponectin for blood sugar level control when re-suspended in water. It is true that ascorbic acid and ingredients of phosphate buffer have been added to such water-suspensions before use as an abundant precaution against inactivation by interaction with other plant juice constituents, including, without limitation, protein-phenol interaction; however, it is not yet verified whether these steps are essential or not. It is also reasonably possible to add dry inert solids which shall lead to make a dry composition; such formulations are also included within the scope of this invention. It has also been noted that concentrated juice solubles also show a white sediment after a period of time, which would be nothing else but the 26 KD or less molecular weight components of the juice soluble which can be separated from the soluble liquid over them by decantation and the sediment can be recovered for use. It may also be possible to recover the precipitate of the proteins below 26 KD molecular weight directly by submitting juice soluble treated to precipitate/insolubilize these proteins directly to decanter centrifuge and collect the sediment.

Metformin replacement by the IC 1 composition: It was experienced that right from the first day of consumption of this IC 1 composition or by suspending the sediment collected from this 6 times concentrated liquid concentrate to make a liquid composition, which with respect to the original juice form which the sediment was derived was 100 times concentrated composition (100 LC), the feeling of hunger returned much earlier than usual on all the three times of the day, stimulating more consumption of food to get the satiety. However, despite satiety after eating more food, the experience was that in the afternoon and in the evening, fatigue was being experienced while walking. With the knowledge that Metformin acts through its action on cells which slows down release rate of glucose between meals when energy is required for maintenance and for activities, it appeared that if the composition comprising the below 26 KD proteins of juice soluble was scavenging away the excess glucose fast enough to storage sites, return of glucose in metabolic cycle for utilization was perhaps being slowed down by Metformin, and in this context now the dose of Metformin which was not an overdose in the context when IC1 was not being consumed, became an overdose and needed to be scaled down. Hence, it was decided to discontinue one 500 mg Metformin tablet of the evening, out of the three that were being administered every day. For two days, the negative test of urine for all the samples of urine throughout 24 hours continued to be negative. However, thereafter, glucose started occasionally appearing after the lunch in the urine to at least about 0.5% of glucose in urine as indicated by Benedict's reagent test, indicating that the post-prandinal sugar is remaining at a level little above the renal threshold, sometimes below and occasionally going a little above the threshold and appearing in the urine. However, instead of restoring the Metformin, it was explored whether increase in the dosage of the 100 LC composition and frequency of consumption of the composition to four times a day would compensate for the full effect of the omitted Metformin. The timing of administration of Metformin was also changed from earlier schedule of one at breakfast and second at lunch in the afternoon to one with early morning snack of a fruit in early morning about two hours before breakfast and the other one with dinner in the evening, leading to a spacing of about 12 hours between the two Metformin administrations.

During the course of the exploration, the dosage of the improved compositions IC1, IC2, 100 LC was increased from 80 mg per dosage progressively to 160, and 320 mg/dose. However, some appearance of urine sugar continued to occur, however, stepwise withdrawal of all the Metformin tablets, initially the ones in the night and morning and lastly of the dose at lunch was possible and the post prandinal blood sugar level, although exceeded 180 mg/dl, remained fairly lower than the post-prandinal blood sugar level of 224 mg/dl which was recorded on full Metformin dose alone, with one additional post-prandinal sugar record of 238 mg/dl before start of the latest series of administration of the improved compositions of this invention. Two months after altogether stopping the metformin dose, the blood sugar levels was 108.8 mg/dl fasting and 193.5 mg/dl post prandinal and about 11 months afterwards the level was 123 mg/dl fasting and 209 mg/dl post-prandinal. It is noteworthy that even after 11 months after complete stoppage of Metformin, the blood sugar levels did not cross the blood sugar levels of 138 mg/dl fasting and 224 mg/dl post-prandinal; giving an evidence thereby that efficacy of the improved composition was better than Metformin and could replace the same.

Just when it looked as if this provided an uppermost efficacy level of the improved composition, immediate release tablets containing 40 mg and 80 mg of the improved composition 7F and slow release tablet of 320 mg made from 7F were made and evaluated. The slow release 320 mg tablets were first evaluated by Subject 1 by consuming twice in 24 hours at 12 hours interval. However, no perceptible improvement was noted. Thereafter, 40 mg tablets were evaluated four times a day (one each after getting up, at breakfast, lunch and early night bed time snack); and it was surprising that after a long time, again the pooled urine sugar tested negative at all the four intervals, indicating that it was better at optimum release of active ingredient when compared to the syrups (IC 1, IC 2 and 100 LC and 320 mg slow release tablet. Following this finding, after consuming 40 mg tablets for four days consecutively, the dose was reduced to half a tablet of 40 mg each time; and that also helped in maintaining the urine sugar level to zero at all the times. The last evaluation was done for 80 mg tablets, four times a day at the times indicated for 40 mg tablets, and the observations were same as for 40 mg tablets. All this indicates that the threshold level for efficacy may be 40 mg or lower, and there is scope for identifying the correct optimum dose and also for further exploring ways for more efficient release of active agent so that the dose can be further reduced.

The HBA1C count had worsened for the Subject 1 to 9.56 in 2013 when the patient was being administered with Metformin alone, the same was around 8.6 in 2016 when the improved compositions of this invention were started to be administered, was, about 11 months after complete stopping of Metformin and with sole use of the improved composition of this invention was 8.16. With the fact that the tablets are seen to be more efficient in optimum release of the active as indicated by negative test on urine sugar at all the times, even by quantitative Benedicts reagent test, this treatment may help in bringing the slight improvement in HBA 1C count to bring it to an acceptable level for a 65 year old adult who is diabetic at least for last eleven years.

HDL cholesterol levels increased from 30 mg/dl to 50 mg/dl.

It was also observed that when the administration of the improved composition was started in March, 2016, the hypertension was also on slow rise to a tune of 160/90 in each evening resulting in headache. This got reduced to around 140/80, and soon to 130/72 forcing reduction of the Amlodipine dose from 5 mg per day to 2.5 mg per day.

Fatigue which was progressively increasing in 2016 to a chronic level also disappeared within one month of starting the treatment of the improved composition in March 2016 and quality of life improved.

Thus, there was an all round improvement in the health status of the Subject no. 1.

In a Subject no. 2, a woman aged 58 years, who also has been mentioned by Savangikar and Savangikar (2014), the blood sugar levels as indicated by urine sugar levels remained within control (144 mg/dl fasting and 207 mg/dl post prandinal by administration throughout the year with the above mentioned compositions of this invention totally replacing Metformin treatment used earlier.

The levels for adults.

In a third subject, who got diagnosed with glucose intolerance was administered with all the above described compositions at 80 mg per dose twice a day; once with breakfast and next in the night with dinner. His blood sugar levels have returned to normal level (105 mg/dl fasting and 129 mg/dl post-prandinal.

1. In a method of using for anti-pathogenesis effect an improved composition comprising heat stable plant juice soluble plant proteins; the improvement comprising that the composition comprising heat stable plant juice soluble plant protein is essentially a water insoluble concentrated mass free from components more soluble in the plant juice, and enriched in heat stable proteins of the plant juice.

2. An improved composition of heat stable plant juice soluble plant proteins; wherein the improvement comprising in (a) essentially water insoluble property of the concentrate mass, (b) the concentrated mass being essentially free from components more soluble in the plant juice, and (c) being enriched in heat stable proteins in comparison with composition of plant juice solubles; wherein
the composition comprising heat stable plant juice soluble plant protein is essentially a water insoluble concentrated mass free from components more soluble in the plant juice, and enriched in heat stable proteins of the plant juice.

3. The composition of claim 2 further comprising dosage forms made from the composition of claim 2.

4. The method of using composition of claim 1 for anti-pathogenesis effect; wherein the composition is standardized for a parameter of a property of the composition that is relatable to anti-pathogenesis property or to the content of the protein molecules having molecular weight of 26 kilo Daltons (kD)or less.

5. The method of using the composition of claim 1 wherein the anti-pathogenesis effect comprises an anti-microbial effect on living beings including microbes or as an Adiponectin agonist effect in mammals.

6. The method of using the composition of claim 3 wherein the Adiponectin agonist effect in mammals comprises reducing the risk of one or more of a disorder selected from Type II diabetes, hyperlipidemia, atherosclerosis, hypertension, heart disease and obesity.

7. The method of using the composition of claim 2 wherein the anti-microbial effect comprises reducing the risk of or treating the microbial infections in plants and animals, with or without synergistic interaction with other antimicrobial agents.

8. An improved antipathogenesis composition comprising heat stable plant juice soluble plant proteins, wherein the heat stable plant juice soluble plant proteins are obtained as juice solubles by subjecting plant or plant juice to heat treatment to precipitate proteins, overwhelming excess of which are heat coagulable, along with a group of phytochemicals which co-coagulate with the heat precipitable proteins in plants leaving behind other plant constituents carrying a small quantity of heat stable juice soluble proteins in the juice to yield juice solubles, and wherein the composition is standardized for a parameter of a property of the composition that is relatable to the anti-pathogenesis property or to the content of the protein molecules having molecular weight of 26 kilo Daltons (kD) or less; the improvement comprising that the composition comprising heat stable plant juice soluble plant protein is essentially a water insoluble concentrated mass free from components more soluble in the plant juice, and enriched in heat stable proteins of the plant juice.

9. The composition of claim 8 wherein the standardization for a parameter of the property of the composition is determined in terms of one or more of the analytical parameters selected from the group comprising: (a) dry matter percentage of the composition, (b) nitrogen percent of dry matter of the composition, (c) content in terms of casein or gallic acid, (d) by molecular weight determination, or by (e) Forurier Transform Infra Red spectra.

10. The composition of claim 8 wherein the heat stable plant juice soluble plant proteins are used with or without pharmaceutical actives, pharmaceutical excipients, cosmaceutical actives, cosmaceutical excipients, nutraceutical actives, nutraceutical excipients, plant protection actives, food additives or other food ingredients.

11. A method/process of making a composition comprising heat stable plant juice soluble plant proteins the process comprising steps of:
a. heat precipitating a plant juice and separating the macromolecules including proteins that coagulate on heating from soluble constituents to get juice solubles containing heat stable plant juice soluble plant proteins,
b. quantifying the heat stable plant juice soluble plant proteins by a method that is relatable to the anti-pathogenesis property or to the content of the protein molecules having molecular weight of 26 kilo Daltons or less,
c. concentrating or diluting to reach the figure of the parameter of a property that is targeted/chosen as standard, and,
d. further optionally adding additive ingredients selected from the group comprising pharmaceutical actives, pharmaceutical excipients, cosmaceutical actives, cosmaceutical excipients, nutraceutical actives, nutraceutical excipients, plant protection actives, food additives or other food ingredients;
wherein,
the composition comprising heat stable plant juice soluble plant protein is essentially a water insoluble concentrated mass free from components more soluble in the plant juice, and enriched in heat stable proteins of the plant juice.

12. The composition of claim 3 wherein the dosage forms comprise concentrated syrup, chewable or orally dissolving tablets, granules, powder, immediate delivery tablets, intermediate delivery tablets, controlled delivery tablets, capsules, orally dissolvable films with or without microemulsion of the improved composition.

13. A composition essentially characterized by Infra-red absorption spectrum comprising a peak at around 3365 wavelength having % transmittance of between 35-60, a peak at around wavelength 2975 having % transmittance of between 60-80, a peak at around wavelength 1612 having % transmittance of between 60-80, a peak at around wavelength 1306 having % transmittance of between 65-70, a peak at around wavelength 1280 having % transmittance of between 70-90, a peak at around wavelength 1080 having % transmittance of between 20-40, a peak at around wavelength 887 having % transmittance of between 70-98 and a peak at around wavelength 825 having % transmittance of between 85-98;
wherein the composition comprises heat stable plant juice soluble plant protein that is essentially a water insoluble concentrated mass free from components more soluble in the plant juice, and enriched in heat stable proteins of the plant juice.
